(19) United States
(12) Patent Application Publication
(10) Pub. No.: US 2007/0196918 A1
(43) Pub. Date: Aug. 23, 2007

(54) Reprogramming of adult human testicular stem cells to pluripotent germ-line stem cells

(76) Inventors: Chauncey B. Sayre, Irvine, CA (US); Francisco J. Silva, Tustin, CA (US); Kwok-Yuen F. Pau, Irvine, CA (US)

Correspondence Address: KIRKPATRICK & LOCKHART PRESTON GATES ELLIS LLP, 1900 MAIN STREET, SUITE 600, IRVINE, CA, 92614-7319, US

(21) Appl. No.: 11/694,687

(22) Filed: Mar. 30, 2007

Related U.S. Application Data
(63) Continuation-in-part of application No. 11/488,362, filed on Jul. 17, 2006.
(63) Continuation-in-part of application No. 11/279,611, filed on Apr. 13, 2006, now abandoned.
(63) Continuation-in-part of application No. 11/060,131, filed on Feb. 16, 2005.
(60) Provisional application No. 60/743,996, filed on Mar. 30, 2006.
(60) Provisional application No. 60/699,680, filed on Jul. 15, 2005.
(60) Provisional application No. 60/671,826, filed on Apr. 14, 2005.
(60) Provisional application No. 60/588,146, filed on Jul. 15, 2004.

(51) Int. Cl. C12N 5/08; C12N 15/09

(52) U.S. Cl. 435/366; 435/371; 435/455

(57) ABSTRACT

Methods for therapeutically programming human adult stem cells into pluripotent cells are provided. Cell therapeutically programmed from adult testicular cells are disclosed. The therapeutically reprogrammed cells are suitable for cellular regenerative therapy and have the potential to differentiate into more committed cell lineages.

### CROSS-REFERENCE TO RELATED APPLICATIONS

The present application claims the benefit under 35 U.S.C. §119(e) to U.S. Provisional Patent Application No. 60/743,996 filed Mar. 30, 2006 and is a continuation-in-part of U.S. patent application Ser. No. 11/488,362 filed Jul. 17, 2006, which claims the benefit under 35 U.S.C. §119(e) of 60/699,680 filed Jul. 15, 2005, and which in turn is a continuation-in-part of U.S. patent application Ser. No. 11/279,611 filed Apr. 13, 2006 which claims the benefit under 35 U.S.C. §119(e) of 60/671,826 filed Apr. 14, 2005 and is a continuation-in-part of U.S. patent application Ser. No. 11/060,1311 filed Feb. 16, 2005 which claims the benefit under 35 U.S.C. §119(e) of 60/588,146 filed Jul. 15, 2004. All the above-referenced applications are incorporated by reference herein in their entirety.

### FIELD OF THE INVENTION

The present invention relates to the field of therapeutically reprogrammed cells. Specifically, human therapeutically reprogrammed cells are provided that are not compromised by the aging process, are immunocompatible and will function in the appropriate post-natal cellular environment to yield functional cells after transplantation.

### BACKGROUND OF THE INVENTION

Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells—blood cells that can develop into several types of blood cells, but cannot develop into brain cells. At the end of the long chain of cell divisions that make up the embryo are “terminally differentiated” cells—cells that are considered to be permanently committed to a specific function.

Scientists had long held the opinion that differentiated cells cannot be altered or caused to behave in any way other than the way in which have had been naturally committed. In recent stem cell experiments, however, scientists have been able to persuade blood stem cells to behave like neurons. Therefore research has also focused on ways to make multipotent cells into pluripotent types (Kanatsu-Shinohara M. et al. Generation of pluripotent stem cells from neonatal mouse testis. Cell 119:1001-12, 2004).s

There are three main groups of stem cells; (i) adult or somatic (post-natal), which exist in all post-natal organisms, (ii) embryonic, which can be derived from a pre-embryonic or embryonic developmental stage and (iii) fetal stem cells (pre-natal), which can be isolated from the developing fetus. Each group of stem cells has their own advantages and disadvantages for cellular regeneration therapy, specifically in their differentiation potential and ability to engraft and function de novo in the appropriate or targeted cellular environment.

In the post-natal animal there are lineage-committed progenitor stem cells and lineage-uncommitted pluripotent stem cells, which reside in connective tissues providing the post-natal organism the cells required for continual organ or organ system maintenance and repair. These cells are termed somatic or adult stem cells and can be quiescent or non-quiescent. Typically adult stem cells share two characteristics: (i) they can make identical copies of themselves for long periods of time (long-term self renewal); and (ii) they can give rise to mature cell types that have characteristic morphologies and specialized functions.

Much of the understanding of stem cell biology has been derived from hematopoietic stem cells and their behavior after bone marrow transplantation. There are several types of adult stem cells within the bone marrow niche, each having unique properties and variable differentiation ability in relation to their cellular environment. Somatic stem cells isolated from human bone marrow transferred in utero into pre-immune sheep fetuses have the ability to xenograft into multiple tissues. Also within the bone marrow niche are mesenchymal stem cells, which have a wide range of non-hematopoietic differentiation abilities, including bone, cartilage, adipose, tendon, lung, muscle, marrow stroma, and brain tissues. In addition, neural stem cells, pancreatic, muscle, adipose, ovarian and spermatogonial stem cells have been found. The therapeutic utility of somatic or post-natal stem cells has been demonstrated and realized through the use of bone marrow transplants. However, adult somatic stem cells have genomes that have been altered by aging and cell division. Aging results in an accumulation of free radical insults, or oxidative damage, that can predispose the cell to forming neoplasms, reduce cell differentiation ability or induce apoptosis. Repeated cell division is directly related to telomere shortening which is the ultimate cellular clock that determines a cell's functional life-span. Consequently, adult somatic stem cells have genomes that have sufficiently diverged from the physiological prime state found in embryonic and prenatal stem cells.

Unfortunately, virtually every somatic cell in the adult animal's body, including stem cells, possesses a genome ravaged by time and repeated cell division. Thus until now the only means for obtaining stem cells having an undamaged, or prime state physiological genome, was to recover stem cells from aborted embryos or embryos formed using in vitro fertilization techniques. However, scientific and ethical considerations have slowed the progress of stem cell research using embryonic stem cells. Generation of embryonic stem cell lines had been thought to provide a renewable source of embryonic stem cells for both research and therapy but recent reports indicate that existing cell lines have been contaminated with immunogenic animal molecules

Another problem associated with using adult stem cells is that these cells are not immunologically privileged, or can lose their immunological privilege after transplant. (The term “immunologically privileged” is used to denote a state where the recipient's immune system does not recognize the cells as foreign). Thus, only autologous transplants are possible in most cases when adult stem cells are used. Thus, most presently envisioned forms of stem cell therapy are essentially customized medical procedures and therefore economic factors associated with such procedures limit their wide ranging potential. Additional barriers to the use of currently available

Moreover, stem cells must be induced to mature into the organ or cell type desired to be useful as therapeutics. The factors affecting stem cell maturation in vivo are poorly understood and even less well understood ex vivo. Thus, present maturation technology relies on serendipity and biological processes largely beyond the control of the administering scientist or recipient.

Current research is focused on developing embryonic stem (ES) cells as a source of totipotent or pluripotent immunologically privileged cells for use in cellular regenerative therapy. However, since embryonic stem cells themselves may not be appropriate for direct transplant as they form teratomas after transplant, they are proposed as “universal donor” cells that can be differentiated into customized pluripotent, multipotent or committed cells that are appropriate for transplant. Additionally there are moral and ethical issues associated with the isolation of embryonic stem cells from human embryos.

Among all adult stem cells, only germ-line stem cells (GSC) retain the ability to transmit genetic information to offspring. Therefore, GSCs are considered a good source of adult stem cells for generation of pluripotent cell lines for therapeutic purposes because of their quiescent state and flexible genome to undergo epigenetic changes.

Like all adult stem cells, spermatogonial stem cells (SSCs) do not normally cross their lineage barrier; their natural path is to produce sperm. However, the reprogramming of SSCs to become pluripotent germ-line stem cells (GSCs) is desirable because of the genomic integrity and reproductive superiority of SSCs.

Therefore, there is a need for sources of biologically useful, pluripotent stem cells having genomes in a nearly physiologically prime state. Furthermore, there is a need for sources of biologically useful, pluripotent stem cells having genomes in a nearly physiologically prime state that maintain their immunological privilege in recipients for a time period sufficient to be therapeutically useful.

### SUMMARY OF THE INVENTION

The present invention provides biologically useful pluripotent therapeutically reprogrammed cells, generated from adult human stem cells which are suitable for therapeutic applications.

Gonadal stem cells, with uncompromised genomic integrity, may be an ideal stem cell source for cell replacement therapy and therapeutic cloning after reprogramming to pluripotent germ-line stem cells (GSCs). In male post-natal mice, spermatogonial stem cells (SSCs) isolated from the testis can be reprogrammed to form GSC cell lines. GSCs exhibit pluripotent markers, differentiate into embryoid bodies and cells of all three germ layers in vitro, and form chimeric cell populations after incorporation into mouse embryos. Moreover, GSCs contain long telomerase repeats; they are self-renewing and do not form teratomas after transplantation.

In one embodiment of the present invention, a method is provided for therapeutic reprogramming comprising isolating a human adult stem cell; contacting said human adult stem cell with a medium comprising stimulatory factors which induce development of said stem cell into a therapeutically reprogrammed cell; recovering said therapeutically reprogrammed cell from said medium.

In another embodiment of the present invention, the human adult stem cell is isolated from the testes. In another embodiment, the human adult stem cell is a spermatogonial stem cell. In another embodiment, the medium comprises PM-10™ medium.

In another embodiment, the therapeutically reprogrammed cell is matured into a more terminally differentiated cell. In yet another embodiment, the more terminally differentiated cell is a cardiac myocyte. In another embodiment, more terminally differentiated cell is a neural cell.

In an embodiment of the present invention, the method further comprises the step of culturing the therapeutically reprogrammed cell to form a cell line.

In another embodiment, the method further comprises the step of implanting the therapeutically reprogrammed cell, or a cell matured therefrom, into a host in need of a therapeutically reprogrammed cell.

In one embodiment of the present invention, a pluripotent therapeutic composition is provided comprising a therapeutically reprogrammed human adult stem cell. In another embodiment, the human adult stem cell is isolated from the testes. In another embodiment, the therapeutically reprogrammed human adult stem cell is produced according to the therapeutic reprogramming method.

In one embodiment of the present invention, a pluripotent therapeutic composition is provided comprising a therapeutically reprogrammed human adult stem cell which has been induced into a more terminally differentiated cell prior to implantation into a host in need of a therapeutically reprogrammed cells.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 depicts the morphologies of adult human testicular isolates in culture on ultra-low adhesive dishes (A), gelatin-coated dishes (B), fibronectin-coated dishes (C) or mouse embryonic feeder cell (MEF)-coated dishes (D) after culture for different periods of time (panels 1-4) in serum-free PM-10™ medium according to the teachings of the present invention.

FIG. 2 depicts the expansion of a reprogrammed adult human testicular stem cell (AHTSC) population in the presence of serum and expression of pluripotent stem cell markers Oct-4 and Nanog according to the teachings of the present invention. FIG. 2A: AHTSCs plated on gelatin and cultured for 46 days in serum-free PM-10™ medium contained colonies (arrows and insert) growing on spindle-like cells; the scale bar is 200 μm; FIG. 2B: Rapid cell growth was observed after 2 days of serum addition; FIG. 2C: The proliferation rate of AHTSCs is depicted in the presence of serum (▪) or serum replacement (•). Many cells in the expanded AHTSC population expressed Oct-4 (FIG. 2D) and Nanog expression (FIG. 2E). Scale bar is equivalent to 50 μm.

FIG. 3 depicts stem cell markers expressed in a AHTSC population with normal karyotype according to the teachings of the present invention. FIG. 3A: Profile of surface markers on AHTSCs that resemble profile on spermatogonial and mesenchymal stem cells, but not on hematopoetic stem cells. Open histograms indicate appropriate isotype control, shaded histograms depict specific antibody staining. Numbers identify the percentage of positive cells. FIG. 3B: Expression of pluripotent stem cell and germ line-specific (DAZL) genes. GAPDH is control for RT-PCR. nt=no template, c=control, monkey embryonic stem cells, p0=passage 0. FIG. 3C: Expression of Oct-4, Nanog and germ-line specific gene Stellar persists throughout several passages (p0, p3 and p5). nt=no template, c=control, human embryonic stem cells. FIG. 3D: After 5 passages AHTSC maintained a normal karyotype.

FIG. 4 depicts the expression of pluripotent (Oct-4, Nanog, Dppa5 and Rex-1), gonadal (DAZL) and control (β-actin) genes in testicular tissues and isolates by reverse transcriptase-polymerase chain reaction (RT-PCR) before and during culture (between 17 and 42 days) according to the teachings of the present invention.

FIG. 5 depicts the expression of pluripotent markers Oct-4 (FIG. 5A), Nanog (FIG. 5B), Oct-4+Nanog (FIG. 5C), alkaline phosphatase (FIG. 5D), TRA-1-60 (FIG. 5E) and human mitochondrial protein (FIG. 5F) by spermatogonial stem cells during reprogramming at four weeks after isolation according to the teachings of the present invention.

FIG. 6 depicts the spontaneous differentiation of spermatogonial stem cells into cardiomyocytes after more than 30 days in culture and expression of cardiac specific markers troponin-1 (FIG. 6A), cardiac myosin (FIG. 6B), cardiac α-actin (FIG. 6C) and cardiac α-actin and human mitochondrial protein (hMP) according to the teachings of the present invention. FIG. 6E depicts the expression of markers by differentiated cells determined by RT-PCR.

FIG. 7 depicts the culture of testicular cells on fibronectin-coated surfaces for more than 30 days and their spontaneous differentiation into neural cells according to the teachings of the present invention. The cells were assayed for expression of neural-specific markers including MAP-2C (FIG. 7A), NF-160 (FIG. 7B), GFAP and hMP (FIG. 7C-D at two magnifications), Oil Red (FIG. 7E) and nestin (FIG. 7F). FIG. 7G depicts the expression of markers by differentiated cells determine by RT-PCR.

FIG. 8 depicts AHTSCs differentiated into mesodermal and endodermal lineages in vitro according to the teachings of the present invention. FIG. 8A: Osteogenic induction: Alizarin Red staining of control (left image) and induced AHTSCs (19 days after induction, right image). Scale bar is 400 μM. FIG. 8B: Expression of osteo-specific genes in AHTSCs after induction. FIG. 8C: Chondrogenic induction: Alcian Blue staining of control (left image) and induced AHTSCs (13 days after induction, right image). Scale bar is 400 μM. FIG. 8D: Expression of chondro-specific genes in AHTSCs after induction. FIG. 8E: Adipogenic induction: Oil Red staining of control (left image) and induced AHTSCs (22 days after induction, right image). Scale bar is 25 μM. FIG. 8F: Expression of hepatocyte-specific genes in AHTSCs after induction. FIG. 8G: Cardiogenic induction: confocal images of immunofluorescent staining with antibodies specific to cardiocytes after 38 days of induction. Scale bar is 100 μM. FIG. 8H: Expression of cardio-specific genes in AHTSCs after induction.

FIG. 9 depicts the differentiation of AHTSCs into the neural lineage cells in vitro according to the teachings of the present invention. FIG. 9A: AHTSCs expressed progenitor (nestin), neuronal (Tuj-III/β-tubulin-3, MAP2, NeuN, NFL, NF160) and glial (GFAP, MBP, GalC) markers after neural induction protocol as assayed by immunocytochemistry. Scale bar for all images is 100 μM. FIG. 9B: Expression of gene markers characteristic of neuronal and glial phenotypes after neural protocol induction as determined by RT-PCR; nt=no template; c=positive control; n=non-induced cells; i=induced cells.

FIG. 10 depicts the induction of neurogenesis in adult human testicular isolates by growth factors critical for embryonic neuroectoderm formation according to the teachings of the present invention. Immunohistochemistry was performed on cells cultured with sonic hedgehog (SHH), fibroblast growth factor 8 (FGF-8) and platelet derived growth factor-BB (PDGF-BB) and with (FIGS. 10A and 10C) or without (FIGS. 10B and 10D) transforming growth factor β (TGF-β). FIG. 10E depicts the expression of markers by differentiated cells determined by RT-PCR.

FIG. 11 depicts transplanted AHTSCs identified by the staining of a human-specific nucleic protein (HuNu) in the spinal cord of uninjured NOD/SCID mice 21 days after transplantation according to the teachings of the present invention. The upper panel shows AHTSCs in the white matter that were double stained with HuNu (labeled with AlexaFluor® 488) and Tuj-III (labeled with AlexaFluor® 568) (FIGS. 11A-B). The lower panel shows grey matter with transplanted AHTSCs (HuNu) that did not express the astroglial marker GFAP (FIGS. 11C-D). Scale bars are 50 μM (FIGS. 11A and 11C) and 20 μM (FIGS. 11B and 11D). The arrows point to double-stained HuNu+/Tuj-III+AHTSCs

FIG. 12 depicts AHTSCs in injured spinal cord of NOD/SCID mice 35 days after transplantation according to the teachings of the present invention. Transplanted human cells were identified by immunofluorescence staining with an antibody against human nuclei (HuNu, FIG. 12A-H). Confocal microscopy demonstrates the double labeling of HuNu with oligodendrocyte markers NG2 (FIG. 12A-B) and GalC (FIG. 12C-D) and neuronal markers Tuj-III (FIG. 12E-F) and MAP-2 (FIG. 12G-H), all in red. Arrows indicate double-stained AHTSCs. Scale bar is 50 μM for the left panel and 20 μM for the right panel.

FIG. 13 depicts AHTSCs transplanted into the injured spinal cord of NOD/SCID mice express the neuronal/oligodendroglial progenitor marker A2B5 according to the teachings of the present invention. FIG. 13A: Double staining of AHTSCs with HuNu antibody (examples are indicated by arrows) and DNA dye TO-PRO-3. Mouse cells showed DNA stain only. FIG. 13B: TO-PRO-3 staining only of the same field as in FIG. 13A showing that AHTSCs have large nuclei (arrows) without bright speckles, a prominent attribute of mouse nuclei. These characteristics allow distinguishing between human and mouse nuclei using DNA-specific stain. FIG. 13C: Double staining with A2B5 (labeled with AlexaFluor® 488) and TO-PRO-3 showing localization of A2B5 in human cells (arrows). FIG. 13D: TO-PRO-3 stain only of the same field as in FIG. 13C showing human (examples are indicated by arrows) and mouse nuclei. Scale bar for all images is 20 μM

FIG. 14 depicts AHTSCs transplanted into the injured spinal cord of NOD/SCID mice which express neuronal marker Tuj-III and increase Tuj-III expression on host mouse cells at the injury site according to the teachings of the present invention. Control mice received either human foreskin fibroblasts (HFF) or vehicle. FIG. 14A: Transplanted AHTSCs identified by anti-Human Mitochondria (HM) antibody (labeled with AlexaFluor® 488) co-expressed Tuj-III (labeled with AlexaFluor® 568). FIG. 14B: Transplanted HFF did not show Tuj-III co-expression. FIG. 14C: Mouse cells expressed elevated levels of Tuj-III around the epicenter of the injury site (indicated by dotted lines) in spinal cord transplanted with AHTSCs in comparison with HFF (FIG. 14D). Scale bars are 20 μM (FIGS. 14A-B) and 200 μM (FIGS. 14C-D).

FIG. 15 depicts the reduced functional deficits in mice transplanted with AHTSCs into the injured spinal cord of NOD/SCID mice according to the teachings of the present invention. Control mice received either human foreskin fibroblasts (HFF) or vehicle (DMEM). FIG. 15A: Basso Mouse Scoring showing significant differences in functional activity between AHTSC-transplanted cohorts and vehicle control cohorts at 28, 35 and 42 days post-injury (DPI). Transplantation started after 6 days of spinal cord injury as indicated by arrow (Day 7). FIG. 15B: Kinematic assay on hindlimb stride width in experimental animals (the same groups as in FIG. 15A). FIG. 15C: Representative images from hematoxylin and eosin (H&E) stained spinal cords from all three experimental groups showing reduced tissue loss in AHTSCs and HFF transplanted animals. FIG. 15D: Morphometric analysis on tissue sections 1 mm either side of the injury epicenter indicates that the spinal cord size in AHTSC cohorts was significantly different from the vehicle control group. * p<0.05; ** p<0.01

### DEFINITION OF TERMS

The following definition of terms is provided as a helpful reference for the reader. The terms used in this patent have specific meanings as they related to the present invention. Every effort has been made to use terms according to their ordinary and common meaning. However, where a discrepancy exists between the common ordinary meaning and the following definitions, these definitions supersede common usage.

Committed: As used herein, “committed” refers to cells which are considered to be permanently committed to a specific function. Committed cells are also referred to as “terminally differentiated cells.”

Differentiation: As used herein, “differentiation” refers to the adaptation of cells for a particular form or function. In cells, differentiation leads to a more committed cell.

Embryonic Stem Cell: As used herein, “embryonic stem cell” refers to any cell that is totipotent and derived from a developing embryo that has reached the developmental stage to have attached to the uterine wall. In this context embryonic stem cell and pre-embryonic stem cell are equivalent terms. Embryonic stem cell-like (ESC-like) cells are totipotent or pluripotent cells not directly isolated from an embryo. ESC-like cells can be derived from primordial sex cells that have been dedifferentiated in accordance with the teachings of the present invention.

Fetal Stem Cell: As used herein, “fetal stem cell” refers to a cell that is multipotent and derived from a developing multi-cellular fetus that is no longer in early or mid-stage organogenesis.

Germ Cell: As used herein, “germ cell” refers to a reproductive cell such as a spermatocyte or an oocyte, or a cell that will develop into a reproductive cell.

Multipotent: As used herein, “multipotent” refers to cells that can give rise to several other cell types, but those cell types are limited in number. An example of a multipotent cell is a hematopoietic cell—a blood stem cell that can develop into several types of blood cells but cannot develop into brain cells.

Multipotent Adult Progenitor Cells: As used herein, “multipotent adult progenitor cells” refers to multipotent cells isolated from the bone marrow which have the potential to differentiate into mesenchymal, endothelial and endodermal lineage cells.

Pluripotent: As used herein, “pluripotent” refers to cells that can give rise to any cell type except the cells of the placenta or other supporting cells of the uterus.

Pluripotent Germ Stem Cell: As used herein “pluripotent germ stem cell” or “PGS” refers to a primordial sex cell that has been therapeutically reprogrammed to be pluripotent and can be maintained in culture.

Post-natal Stem Cell: As used herein, “post-natal stem cell” refers to any cell that is multipotent and derived from a multi-cellular organism after birth.

Primordial Sex Cell: As used herein, “primordial sex cell” refers to any diploid cell that is derived from the male or female mature or developing gonad, is able to generate cells that propagate a species and contains a diploid genomic state. Primordial sex cells can be quiescent or actively dividing. These cells include male gonocytes, female gonocytes, spermatogonial stem cells, ovarian stem cells, oogonia, type-A spermatogonia, Type-B spermatogonia. Primordial sex cells are also known as germ-line stem cells (GSC).

Primordial Germ Cell: As used herein, “primordial germ cell” refers to cells present in early embryogenesis that are destined to become germ cells.

Reprogramming: As used herein “reprogramming” refers to the resetting of the genetic program of a cell such that the cell exhibits pluripotency and has the potential to produce a fully developed organism.

Somatic Stem Cells: As used herein, “somatic stem cells” refers to diploid multipotent or pluripotent stem cells. Somatic stem cells are not totipotent stem cells.

Therapeutic Reprogramming: As used herein, “therapeutic reprogramming” refers to the process of maturation wherein a stem cell is exposed to stimulatory factors according to the teachings of the present invention to yield either pluripotent, multipotent or tissue-specific committed cells. Therapeutically reprogrammed cells are useful for implantation into a host to replace or repair diseased, damaged, defective or genetically impaired tissue. The therapeutically reprogrammed cells of the present invention do not possess non-human sialic acid residues.

Totipotent: As used herein, “totipotent” refers to cells that contain all the genetic information needed to create all the cells of the body plus the placenta. Human cells have the capacity to be totipotent only during the first few divisions of a fertilized egg.

### DETAILED DESCRIPTION OF THE INVENTION

The present invention provides biologically useful pluripotent therapeutically reprogrammed cells, generated from adult human stem cells are suitable for therapeutic applications.

In an embodiment of the present invention, methods and compositions for therapeutically reprogramming adult human stem cells are provided. Therapeutic reprogramming refers to a maturation process wherein a stem cell is exposed to stimulatory factors according the teachings of the present invention to yield pluripotent, multipotent or tissue-specific committed cells. The process of therapeutic reprogramming can be performed with a variety of stem cells including, but not limited to, therapeutically cloned cells, hybrid stem cells, embryonic stem cells, fetal stem cells, multipotent post-natal stem cells (adult progenitor cells), adipose-derived stem cells (ADSC) and primordial sex cells.

Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of a multipotent cell is a hematopoietic cell—a blood cell that can develop into several types of blood cells, but cannot develop into brain cells. At the end of the long chain of cell divisions that make up the embryo are “terminally differentiated” cells—cells that are considered to be permanently committed to a specific function.

Scientists had long held the opinion that differentiated cells cannot be altered or caused to behave in any way other than the way in which have had been naturally committed. In recent stem cell experiments, however, scientists have been able to persuade blood stem cells to behave like neurons. Therefore research has also focused on ways to make multipotent cells into pluripotent types.

The ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct-4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G₀/G₁ phase of the cell cycle. In mice and rats these gonocytes resume division within a few days after birth to generate spermatogonial stem cells and eventually undergo differentiation and meiosis related to spermatogenesis.

Primordial sex cells are directly responsible for generating the cells required for fertilization and eventually a new round of embryogenesis to create a new organism. Primordial sex cells are not programmed to die and are of a quality comparable to that of an embryonic state.

Embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural environment in the epiblast, they only exist transiently during embryogenesis. Manipulation of embryonic stem cells in vitro has lead to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage.

For the purpose of this discussion, an embryo and a fetus are distinguished based on the developmental stage in relation to organogenesis. The pre-embryonic stage refers to a period in which the pre-embryo is undergoing the initial stages of cleavage. Early embryogenesis is marked by implantation and gastrulation, wherein the three germ layers are defined and established. Late embryogenesis is defined by the differentiation of the germ layer derivatives into formation of respective organs and organ systems. The transition of embryo to fetus is defined by the development of most major organs and organ systems, followed by rapid fetal growth.

Fetal stem cells have been isolated from the fetal bone marrow (hematopoietic stem cells), fetal brain (neural stem cells) and amniotic fluid (pluripotent amniotic stem cells). In addition, stem cells have been described in both adult male and fetal tissues. Fetal stem cells serve multiple roles during the process of organogenesis and fetal development, and ultimately become part of the somatic stem cell reserve.

Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory.

During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromise the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandem-repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress.

There is a history of cellular therapy for the treatment of a variety of diseases but the majority of the use has been in bone marrow transplantation for hematopoietic disorders, including malignancies. In bone marrow transplantation, an individual's immune system is restored with the transplanted bone marrow from another individual. This restoration has long been attributed to the action of hematopoietic stem cells in the bone marrow.

There is increasing evidence that stem cells can be differentiated into particular cell types in vitro and shown to have the potential to be multipotent by engrafting into various tissues and transit across germ layers and as such have been the subject of much research for cellular therapy. As with conventional types of transplants, immune rejection is the limiting factor for cellular therapy. The recipient individual's phenotype and the phenotype of the donor will determine if a cell or organ transplant will be tolerated or rejected by the immune system.

Therefore, the present invention provides methods and compositions for providing functional immunocompatible stem cells for cellular regenerative/reparative therapy.

The therapeutic reprogramming method of the present invention is suitable for reprogramming cells from a variety of animals including, but not limited to, primates, rodents, sheep, cattle, goats, pigs, horses, etc. In one embodiment, the primate is a human.

Therapeutic reprogramming takes advantage of the fact that certain stem cells are relatively easily to obtain, such as spermatogonial stem cells and adipose-derived stem cells, and epigenetically reprograms these cells by exposure to stimulatory factors. These therapeutically reprogrammed cells have changed their maturation state to either a more committed cell lineage or a less committed cell lineage. Therapeutically reprogrammed cells are therefore capable of repairing or regenerating disease, damaged, defective or genetically impaired tissues.

Therapeutic reprogramming uses stimulatory factors, including without limitation, chemicals, biochemicals and cellular extracts to change the epigenetic programming of cells. These stimulatory factors induce, among other results, genomic methylation and/or acetylation changes in the donor DNA.

In one specific embodiment of the present invention, primordial sex cells (PSC) are therapeutically reprogrammed. Primordial sex cells, residing in the lining of the seminiferous tubules of the testes and the lining of the ovaries (the spermatogonia and oogonia, respectively) have been determined to possess diploid (2N) genomes remarkably undamaged by to the effects of aging and cell division. Thus, PSCs possess genomes in a nearly physiologically prime state. A non-limiting example of a PSC particularly useful in an embodiment of the present invention is a spermatogonial stem cell. According to the teachings herein, therapeutically reprogrammed PSC cells are prepared for the maturation process using means similar to that experienced by stem cells present in the developing embryo and fetus during embryogenesis and organogenesis.

In an embodiment of the present invention, spermatogonial stem cells (adult human testicular stem cells), isolated from human males undergoing standard castration surgical procedures and who have been on long-term hormone (estrogen) treatment, are therapeutically reprogrammed by culture in the presence of cell growth promoting and maintenance and maturation factors. A series of culture media have been developed by the present inventors which contain cell growth promoting, maintenance, reprogramming and maturation factors for the therapeutic reprogramming of post-natal stem cells. These media are disclosed in co-pending U.S. patent application Ser. No. 11/488,362 which is incorporated by reference herein for all it contains regarding media. In a non-limiting embodiment, the reprogramming medium is PM-10™. PM-10™ media contains the signals necessary for human spermatogonial stem cells to be therapeutically reprogrammed into pluripotent embryonic stem cell-like cells. The cell growth and maturation factors useful for the therapeutic reprogramming of PSCs using PM-10™ media in a serum-free environment include, but are not limited to, epidermal growth factor (EGF), fibroblast growth factor 2 (FGF2), glial cell derived neurotrophic factor (GDNF) and leukemia inhibitory factor (LIF).

The therapeutically reprogrammed human cells made in accordance with the teachings of the present invention are useful in a wide range of therapeutic applications for cellular regenerative/reparative therapy. For example, and not intended as a limitation, the therapeutically reprogrammed human cells of the present invention can be used to replenish stem cells in mammals whose natural stem cells have been depleted due to age or ablation therapy such as cancer radiotherapy and chemotherapy. In another non-limiting example, the therapeutically reprogrammed human cells of the present invention are useful in organ regeneration and tissue repair. In one embodiment of the present invention, therapeutically reprogrammed human cells can be used to reinvigorate damaged muscle tissue including dystrophic muscles and muscles damaged by ischemic events such as myocardial infarcts. In another embodiment of the present invention, the therapeutically reprogrammed human cells disclosed herein can be used to ameliorate scarring in animals, including humans, following a traumatic injury or surgery. In this embodiment, the therapeutically reprogrammed human cells of the present invention are administered systemically, such as intravenously, and migrate to the site of the freshly traumatized tissue recruited by circulating cytokines secreted by the damaged cells. In another embodiment of the present invention, the therapeutically reprogrammed human cells can be administered locally to a treatment site in need or repair or regeneration.

The present inventors have demonstrated that murine spermatogonial stem cells (SSCs) can be reprogrammed to germ-line stem cells (GSCs) that express GFP (a marker of Oct-4 expression) and pluripotent markers, including Oct-4, Nanog, SSEA-1 and alkaline phosphatase. The GFP⁺ murine GSC cell lines have been propagated without losing the expression of pluripotent markers and telomerase activity. Like ESCs, these murine GSCs (mouse PrimeCell™) form embryoid bodies, differentiate into neural, adipose and cardiac phenotypes, incorporate into the inner cell mass of recipient mouse embryos, and form chimeric cell populations in the heart, lung, liver and brain. Unlike ESCs, murine GSCs do not form teratomas after transplantation into SCID mice, thus increasing the therapeutic potential of this type of therapeutically reprogrammed cell. It has also been determined that reprogramming of SSCs to GSCs involves an up-regulation of Oct-4 gene expression at the time of GSC colony formation.

The present inventors disclose herein the derivation of GSCs from adult human testicular isolates which express pluripotent markers during reprogramming, differentiate into multi-lineages, and form multipotent cell lines. One requirement of stem cell therapy is a constant cell supply. While ES and EG cells multiply indefinitely, adult stem cells have a finite renewal limitation. The present inventors have established human pluripotent cell lines from the gonad that are self-renewal and transplantable.

Adult human testicular tissues were dissociated and cultured in the serum-free PM-10™ therapeutic reprogramming medium containing stem cell growth and reprogramming factors in suspension or on adhesive substrates. Before culture, none of the testicular samples expressed the pluripotent marker, Oct-4. After culture, Oct-4 expression was found in nearly all samples which also expressed Nanog, Dppa5, Rex-1, SSEA-3/4, TRA-1-60/81 and alkaline phosphatase. These cells are capable of differentiating into phenotypes that exhibit signature markers of cardiomyocytes, neural cells, adipocytes, osteocytes and chondrocytes.

The germ-line stem cells of the present application are multipotent, expressing several pluripotent stem cell markers and exhibiting differentiation potential to form lineages of all three germ layers. Furthermore, upon transplantation of neural cell differentiated from adult human testicular stem cells (GSCs) into the site of a spinal cord injury in NOD/SCID mice, the AHTSCs survived, integrated into the host tissue, expressed neuronal and oligodendoglial markers and reduced functional deficits. The transplanted cells do not demonstrate tumorogenic activity.

The following examples are meant to illustrate one or more embodiments of the invention and are not meant to limit the invention to that which is described below.

### EXAMPLE 1

### Therapeutic Reprogramming Culture Medium

A cell culture medium for therapeutically reprogramming human stem cells is provided wherein the cell culture medium comprises a cell culture growth medium base; a plurality of vitamins and minerals and a plurality of cell growth and maturation factors. In one embodiment of the present invention, the cell culture medium is serum free.

The therapeutic reprogramming cell culture medium comprises a plurality of cell growth and maturation factors selected from the group consisting of recombinant human epidermal growth factor, recombinant human fibroblast growth factor 2, recombinant human glial cell derived neurotrophic factor and human leukemia inhibitory factor. The recombinant human epidermal growth factor is present at a concentration of between approximately 10 ng/mL and approximately 40 ng/mL, preferably approximately 20 ng/mL. The recombinant human fibroblast growth factor 2 is present at a concentration of between approximately 1 ng/mL and approximately 120 ng/mL. The recombinant human glial cell derived neurotrophic factor is present at a concentration of between approximately 2 ng/mL and approximately 40 ng/mL, preferably between approximately 10 ng/mL and approximately 20 ng/mL. The human leukemia inhibitory factor is present at a concentration of between approximately 1,000 units/mL and approximately 10,000 units/mL, preferably approximately 1,000 units/mL. In one embodiment, the cell culture medium is PM-10™ (Table 1).

TABLE 1

| | PM-10 ™ Medium | |
|---|---|---|
| Stem Cell Basal Medium | StemPro ®-34 Complete | |
| L-glutamine | 2 | mM |
| MEM vitamins | 1× | |
| MEM non-essential amino acids | 1× | |
| L-ascorbic acid | 0.1 | mM |
| d-biotin | 10 | μg/mL |
| β-estradiol | 50 | ng/mL |
| progesterone | 60 | ng/mL |
| bovine serum albumin Fraction VI | 5 | mg/mL |
| DL-lactic acid | 1 | μg/mL |
| pyruvic acid | 30 | μg/mL |
| D-(+)-glucose | 6 | mg/mL |
| sodium selenite | 30 | nM |
| putrescine | 60 | μM |
| transferrin (holo) | 50 | μg/mL |
| bovine insulin | 20 | μg/mL |
| penicillin/streptomycin | 1× | |
| β-mercaptoethanol | 50 | μM |
| recombinant human epidermal | 20 | ng/mL |
| growth factor (rhEGF) | | |
| recombinant human fibroblast growth | 1 | ng/mL |
| factor 2 (rhFGF2) | | |
| recombinant human glial cell derived | 10 | ng/mL |
| neurotrophic factor (rhGDNF) | | |
| 1,000 units/mL human leukemia | 1000 | U/mL |
| inhibitory factor (LIF) | | |

### EXAMPLE 2

### Therapeutic Reprogramming of Testicular Cells

Adult human testes were obtained from patients between 23-52 years of age. These patients were admitted to a clinic for reasons unrelated to cancerous conditions. Their medical history and condition are not to be disclosed in respect to patient's privacy.

Testicular samples were washed 5 times in cold phosphate-buffered saline (PBS) containing 0.01% EDTA. Seminiferous tubules were dissected, minced, and digested with collagenase/DNase. Dissociated cells were centrifuged and re-suspended in the serum-free PM-10™ medium containing a stem cell medium base, non-essential amino acids, and 19 cell growth-promoting and reprogramming factors, including GDNF. Blood and somatic cells were discarded after overnight differential adhesion. Stem cells in suspension were collected and plated onto culture dishes coated with gelatin, fibronectin, mouse feeder cells, or low-adhesive surface. In average, about 150 million cells per testis were obtained. The plating density was adjusted to approximately 10⁶ cells per 7 cm². Cells and cell aggregates were culture on the PM-10™ medium for 40 days during which time multiple samples were collected for pluripotent marker assays.

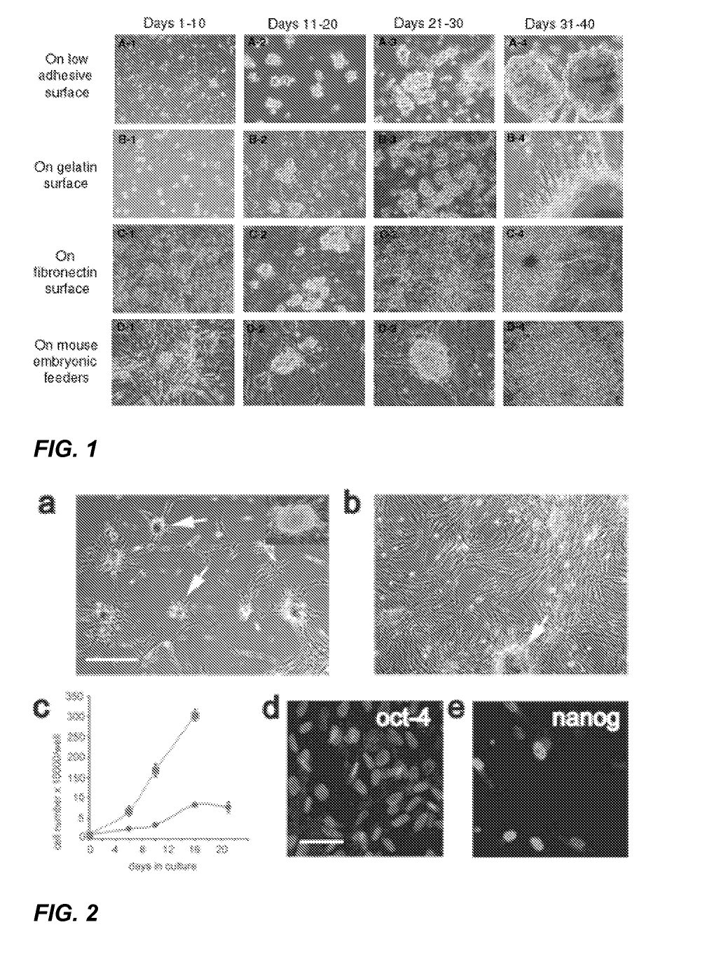

Morphological changes of the isolates representing the first 40 days of culture are chronologically shown in FIG. 1 (panels 1-4). Cells and cell aggregates cultured in Ultra-Low attachment culture dishes (Corning) (A) remained in suspension and grew in size over time as spherical structures (A-3 and A-4). Those that were cultured in gelatin-coated dishes (B) also multiply in suspension for 2-3 weeks before they attached (B-4). In contrast, cells plated onto fibronectin-coated dishes or glass coverslips (C) attached within 2 weeks and grew into colonies (C-3 and C-4). The cells that were co-cultured with mouse embryonic fibroblasts (MEF; as feeder cells) immediately following isolation (D) either differentiated or failed to grow (D-1 and D-2). Addition of fetal bovine serum (20%, D3) or mouse ESC-conditioned medium (30%, D-4) appeared to promote cell growth initially, but the cells in those colonies differentiated within a week and lost their Oct-4 expression.

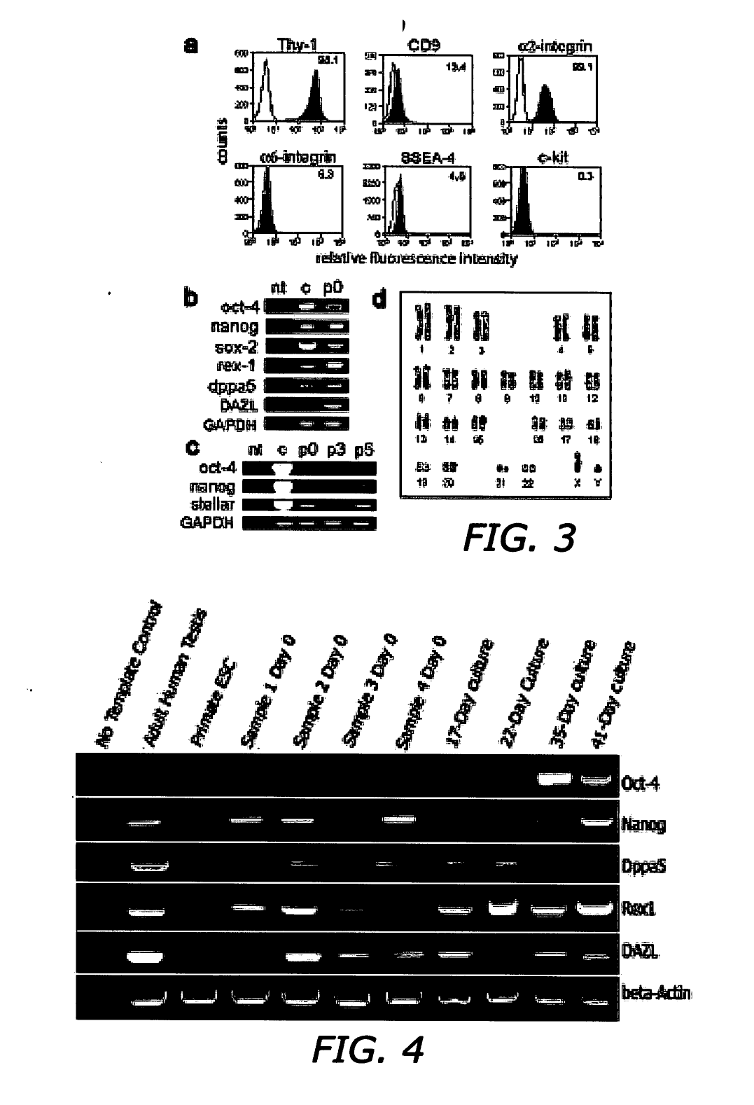

After isolation from adult testes, AHTSCs were maintained in serum-free PM-10™ medium for several months when plated onto either gelatin or fibronectin substrates (FIG. 2A). The morphology of these cultures consisted of colonies (arrows and insert in FIG. 2A) growing on clusters of spindle-like cells apparently as outgrowths of the colonies. Addition of serum or serum replacement to the culture promoted cell division within days (FIG. 2B). The estimated rate of doubling cell numbers was every 26 hours and the AHTSC population was propagated every 10 days (FIG. 2C). The AHTSC population stained positive for the pluripotent markers, Oct-4 (FIG. 2D) and Nanog (FIG. 2E). The propagated cells maintained their marker characteristics for at least 6 passages (see FIG. 3 below). No change of morphology or growth patterns was observed after freezing and thawing for up to 12 passages.

### EXAMPLE 3

### Phenotypic Characterization of Therapeutically Reprogrammed Cells

Several markers found on adult stem cells were highly expressed on the surface of AHTSCs, including Thy-1 and α2-integrin as well as a moderate level of CD-9, α6-integrin and SSEA-4, but not c-Kit (FIG. 3A). Moreover, a weak expression of the major histocompatibility complex, MHC-I, was observed, whereas MHC-II and several hematopoietic stem cell markers were non-detectable. The gene (mRNA) expression profile for the AHTSC population taken at day 6 post-reprogramming (passage 0) showed that the cells expressed pluripotent markers, Oct-4, Nanog, Sox-2, Rex-1 and Dppa5 (FIG. 3B). The origin of lineage was identified by the germ cell markers, DAZL and Stellar (FIG. 3B-C). Cells used for transplantation expressed the pluripotent markers, Oct-4 and Nanog (FIG. 2D-E) and were diploid without chromosomal aberrations as determined by karyotype analysis (FIG. 3D).

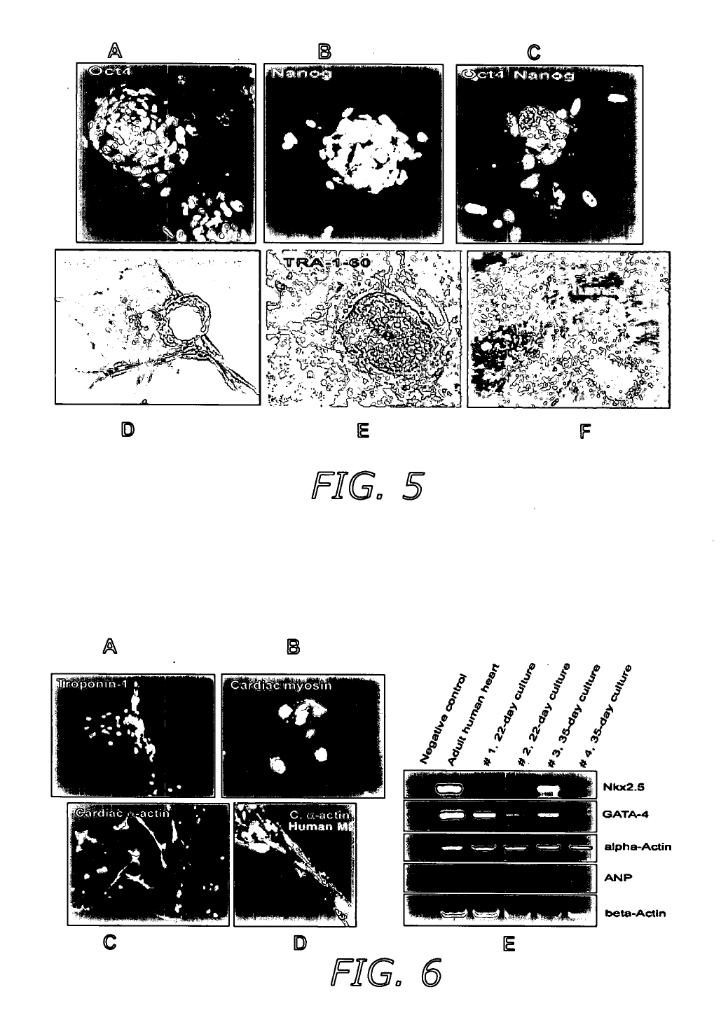

The expression of Oct-4 is a signature of pluripotent cells; its expression disappears as pluripotent cells commit to a cell lineage and differentiate. In a commercially available adult human testicular RNA sample, no Oct-4 expression was found (FIG. 4, lane 2). Similarly in adult human testis samples before therapeutic reprogramming (FIG. 4, lanes 4-7), no Oct-4 expression was detected. In contrast, Oct-4 expression was up-regulated by 17-42 days of culture in suspension or on adhesive surfaces (FIG. 4, lanes 8-11). A non-human primate ESC sample (FIG. 4, lane 3; known to express Oct-4 and Rex-1) was included as positive control. Several markers found in pluripotent ESCs, including Nanog, Dppa5 and Rex-1, were expressed in adult human testicular tissues and isolates (FIG. 4). Morphologically, cells in cultures grown on fibronectin (FIG. 5A-F) and gelatin exhibited pluripotent protein markers by immunocytochemical staining of Oct-4 and Nanog (FIG. 5A-C), alkaline phosphatase (FIG. 5D) and TRA-1-60 (FIG. 5E). The identity of human cells on MEF co-cultures were confirmed by staining the human-specific mitochondria protein (FIG. 5F).

These results demonstrate that Oct-4 gene expression is activated after 17 days or less in PM-10™ medium cultures, and the expression of Oct-4 continues for at least 40 days. As expected, DAZL is expressed in testicular as well as ESC samples.

At various time points during the initial culture period, suspended cells, cell aggregates and/or attached colonies were isolated either manually or by a brief collagenase treatment (1 mg/mL for 15 min). They were then plated onto mouse embryonic feeder cells or fibronectin coverslips surrounded by mouse embryonic feeder cells.

### EXAMPLE 4

### Spontaneous Differentiation of Therapeutically Reprogrammed Cells

Spontaneous differentiation in the suspension cultures and the cells on gelatin or fibronectin surface occurred after approximately four weeks in the serum-free PM-10™ medium. Pluripotent markers were still detected in the culture for 40 days (FIGS. 4 and 5), suggesting that the testicular isolates contain mixed cell populations that respond to the PM-10™ medium in culture differently and at different times. One of the first visible morphological changes in cells grown on gelatin surface is the attachment and formation of colonies that exhibited out-growing processes (see FIG. 1). These cells expressed cardiomyocyte markers troponin-1 (FIG. 6A), cardiac myosin (FIG. 6B), cardiac α-actin (FIG. 6C) and cardiac α-actin and human mitochondrial protein (FIG. 6D), as well as Nkx2.5, GATA-4, cardiac α-actin and myosin, but not atrial natriuretic peptide (ANP) (FIG. 6E). The expression of Nkx2.5, a cardiomyocyte-specific marker, was found when Oct-4 expression was also detected in the culture.

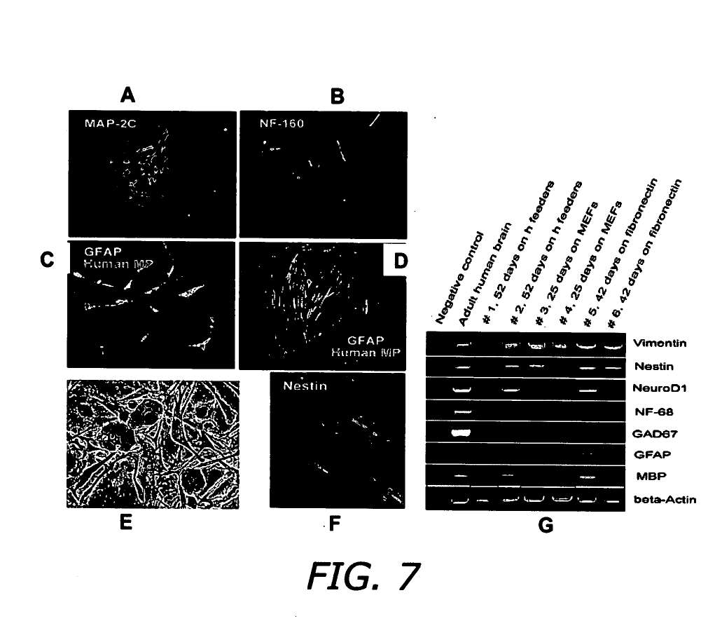

In contrast, cultures grown on fibronectin-coated coverslips showed morphologies different from cardiomyocytes (FIG. 7A-F). These cells attached within 2 weeks and subsequently differentiated into phenotypes that expressed neural lineage markers vimentin, nestin and NeuroD1, as well as neuronal and glial markers, including NF-68/160, MAP-2C, GAD67, GFAP and MBP (FIG. 7G). The expression of MAP-2C (FIG. 7A), NF160 (FIG. 7B), GFAP+hMP (FIGS. 7C and 7D, two magnifications) and nestin (FIG. 7F) were also shown by immunohistochemistry. With the exception of the astroglial marker, GFAP, which is expressed only in cells plated onto fibronectin-coated coverslips (FIG. 7G), the expression of neural progenitor and phenotype markers was found in cells plated onto fibronectin-coated coverslips in dishes covered without or with human feeders or MEFs (the human origin was confirmed by the expression of hMP). Some cells in direct contact with MEFs differentiated into adipocytes with cytoplasmic vesicles exhibited Oil Red staining (FIG. 7E).

### EXAMPLE 5

### Induction of Differentiation in Therapeutically Reprogrammed Cells

The AHTSC population can be induced by neural protocols containing growth factors platelet derived growth factor (PDGF), fibroblast growth factor 2 (FGF-2), epidermal growth factor (EGF), sonic hedgehog (SHH), fibroblast growth factor 8 (FGF-8) and brain derived neurotrophic factor (BDNF) to differentiate into multiple neural phenotypes.

For differentiation protocols, cells were plated onto gelatin-coated (0.2%) plastic dishes or coverslips in DMEM low glucose supplemented with Glutamax, penicillin/streptomycin in the presence or absence of different concentrations of FBS and growth factors according to the specific differentiation protocol as described below.

For neural differentiation, AHTSCs were plated onto fibronectin-coated coverslips and incubated in DMEM/F-12 with the addition of N2 supplements (Invitrogen), PDGF 10 ng/ml, FGF-2 10 ng/ml, and EGF 20 ng/ml for 4-14 days. Control cells on gelatin-coated coverslips were incubated in DMEM with 2% FBS without growth factors.

For induction of AHTSCs into osteocytes, cells were plated onto gelatin-coated dishes and cultured in DMEM low glucose with Glutamax, penicillin/streptomycin, 20% FBS, dexamethazone 100 nM, ascorbic acid 0.25 mM and β-glycerolphosphate 10 mM for up to 25 days.

For chondrocyte induction, AHTSCs were cultured in the Chondrogenic SingleQuots™ media (Cambrex) with the addition of 20% FBS and TGF-3β (10 ng/ml). The induction time was 14 days. The efficiency of differentiation was determined by Alcian Blue stain and RT-PCR for chondrogenic marker expression.

For adipocyte differentiation, cells were plated onto gelatin-coated dishes and cultured during several repeating cycles consisting of induction media for the first 3 days following by maintenance media for 1 day. The induction medium was DMEM with low glucose, Glutamax, penicillin/streptomycin, 20% FBS, dexamethasone 1 μM, isobutylmethylxanthine 0.5 mM, indomethacin 200 μM and insulin 10 μM. The Maintenance medium was DMEM with low glucose, Glutamax, penicillin/streptomycin, 20% FBS and insulin 10 μM. After 20-25 days of induction, successful adipogenesis was confirmed by Oil Red staining.

For hepatocyte differentiation, cells were plated onto Matrigel®-coated plates. The induction medium consisted of DMEM low glucose with MCDB-201 40% (Sigma), Glutamax, penicillin/streptomycin, 5% FBS, and was supplemented with ITS+LA-BSA (10 μg/ml insulin, 5.5 μg/ml human transferrin, 5 ng/ml sodium selenite, 0.5 mg/ml bovine serum albumin, 4.7 μg/ml linoleic acid, dexamethasone 1 nM, ascorbic acid 100 μM, FGF-4 10 ng/ml, and HGF 20 ng/ml. Hepatocyte specific marker expression was identified by PCR

For induction of cardiomyocyte differentiation, PM-10™ base medium (w/o growth factors) with the addition of 20% FBS, 5-AZA-2′-deoxycytidine 4 μM and Cardiogenol C 25 μM was added to cell plated on 0.2% gelatin coverslips. Control conditions were PM-10™ base medium with 20% FBS.

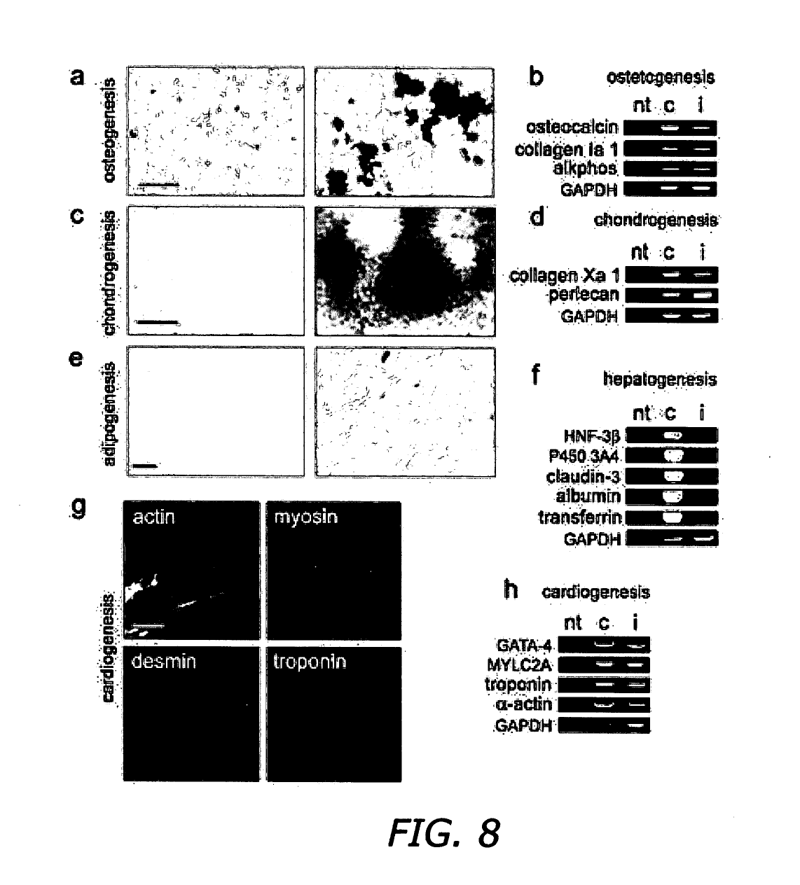

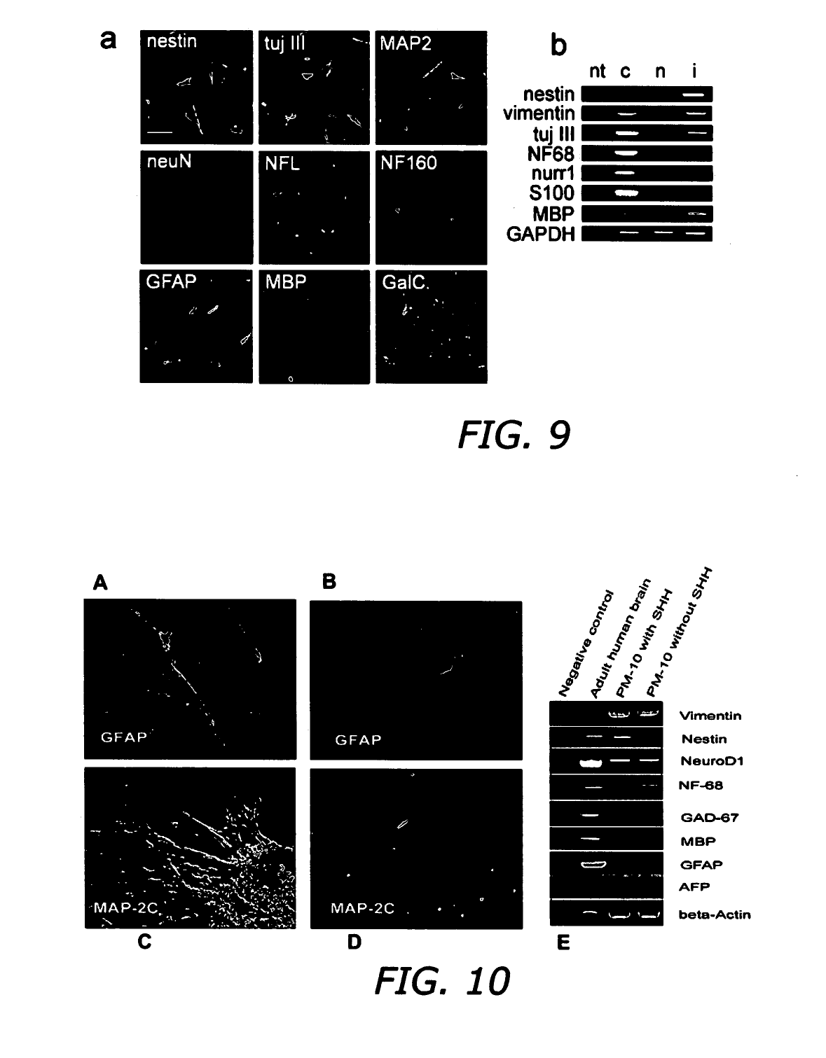

Differentiated AHTSCs expressed markers indicative of neural progenitor cells (nestin, vimentin), neuronal cells (NeuN, NF-L, and NF160, tuj-III/β-tubulin3 and MAP2), glial cells (GFAP, MBP, GalC), but not maturing Schwann cells (S-100) (FIG. 9A-B). Moreover, some cells expressed the dopaminergic marker, Nurr-1. In addition to the ectodermal lineage, AHTSCs can also be induced to differentiate into phenotypes of the mesodermal and endodermal lineages expressing markers that are indicative of cardiomyocytes, chondrocytes, osteocytes, adipocytes, or hepatocytes (FIG. 8).

Testicular isolates spontaneously differentiated into neural cells when plated onto fibronectin substrates but rarely on gelatin surfaces. However, cells on gelatin could be induced to differentiate into neural phenotypes after 30 days of treatment with sonic hedgehog (SHH), fibroblast growth factor 8 (FGF-8), transforming growth factor β (TGF-β) and platelet derived growth factor BB (PGDF-BB) (FIGS. 9 and 10). Cells were cultured in PM-10™ medium with growth factor supplements on the second day after differential adhesion. The supplement of growth factors includes sonic hedgehog (150 ng/ml), fibroblast growth factor (FGF)-8 (75 ng/ml), platelet-derived growth factor (PDGF)-BB (20 ng/ml), and transforming growth factor (TGF)-β3 (4 ng/ml); some are involved in neural progenitor cell formation in the isthmus (or the mid-hindbrain boundary). The cells were cultured in this medium for 40 days with medium changes every other day. The effect of SHH and/or FGF-8 may be critical for glial cell maturation since cells cultured in the control medium with TGF-β and PDGF-BB but without SHH and FGF-8 did not express the glial markers GFAP and MBP (FIG. 10E).

Collectively, these results suggested that adult human testicular isolates containing SSCs can be cultured in a serum-free medium for at least 40 days. They expressed the pluripotent cell marker Oct-4 only after culture, suggesting that reprogramming of SSCs to pluripotent GSCs occurred. Depending on the substrate, some cells spontaneously differentiated toward the adipose, cardiac and neural lineages, which can also be induced by known cardiogenic and neurogenic reagents. The reprogrammed cells are pluripotent GSCs as they can give rise to cells from all three germ layers; cardiomyocytes from the mesodermal germ layer, neural cells from the ectodermal germ layer, and gonadal cells from the endodermal germ layer. Previously, only pluripotent cells, such as ES cells or embryonal germ (EG) cells, were known to be capable of differentiating into all three germ layers.

### EXAMPLE 6

### Transplantation of Therapeutically Reprogrammed Cells Following Spinal Cord Injury

All animal work was performed according to the guidelines established by IACUC written by the Institute of Laboratory Animal Resources with Governing Board of the National Research Council (published by National Academy Press 1996 ISBN 0-309-05377-3) and by the Office of Laboratory Animal Welfare 2002.

Crush injuries were performed under Avertin anesthesia (0.6 ml/20 g). Age matched female NOD/SCID mice (Taconic) were used for all animal studies. A midline incision of the skin was made over T6-L2 and the paravertebral muscles were separated from the T8-T10 vertebrae. Following a T9 dorsal laminectomy, the column was stabilized, and a T9 crush injury was performed by stabilizing the vertebral column, grasping the spinal cord with a fine forceps (FST) calibrated to 0.4 mm and holding the forceps closed for 5 seconds. Animals were placed on soft bedding over a heating pad held at 37° C. for 3 hours and given nourishment. Animals were behaviorally assessed 1 and 6 days following spinal cord injury and randomized to receive cellular transplantations.

All mice (n=27) were acclimated prior to any behavioral testing. Since the characteristics of locomotor recovery are different in mice than in rats, the recently developed locomotor rating scale, the Basso Mouse Scale (BMS) was used. It was developed specifically for mice as a systematic, in-depth analysis of locomotor recovery from SCI. The scale is from 0 to 9, with 0 being no ankle movement and 9 being frequent or consistent plantar stepping, mostly coordinated, paws parallel at initial contact and lift off, normal trunk stability, and tail always up. For kinematic analysis, animals were videotaped using a Canon Digital Video Camcorder ZR500 from underneath plexiglass bearing defined 1 cm grid lines. The videos were analyzed frame by frame using Microsoft Windows Movie Maker software. Analysis consisted of rear paw stride length, defined as the distance from the start of a step with the rear paw thru to the end of that step with the same paw (measurements taken on each side for three consecutive steps and averaged), and stride width, defined as the distance from the left outermost hind paw digit to the right outermost hind paw digit as previously described⁴⁰. All behavioral analysis was conducted one day prior to injury, then on days 1, 6, 8, 14, 21, 28, 35, and 42 following spinal cord injury. All behavioral analysis was done by scorers blinded to the different transplantation groups. One and 6 days following SCI animals were BMS assessed and randomized to receive cellular transplantation. Transplantation groups (n=6 per group) received either human foreskin fibroblasts, adult human testicular stem cells (AHTSC), or DMEM vehicle control 7 days following SCI.

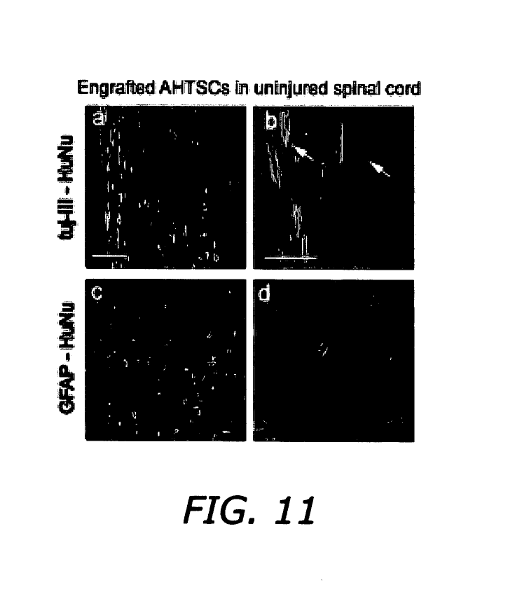

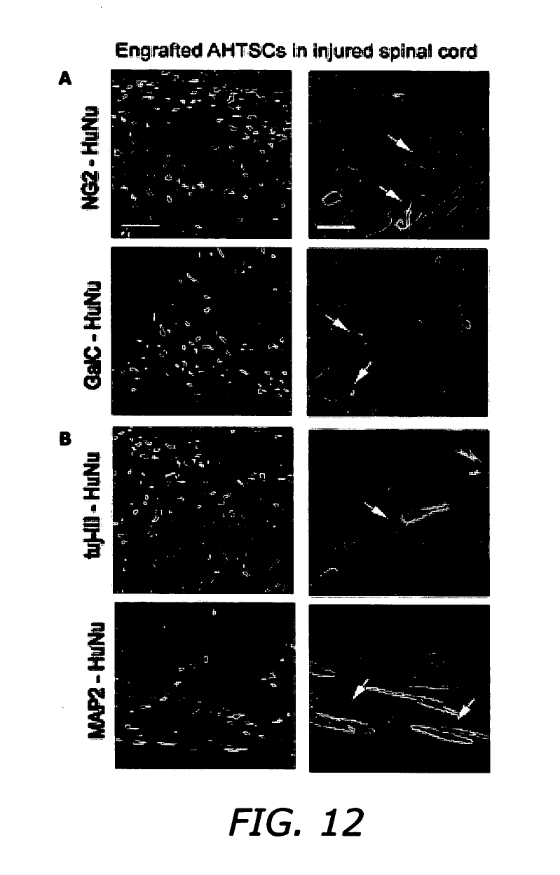

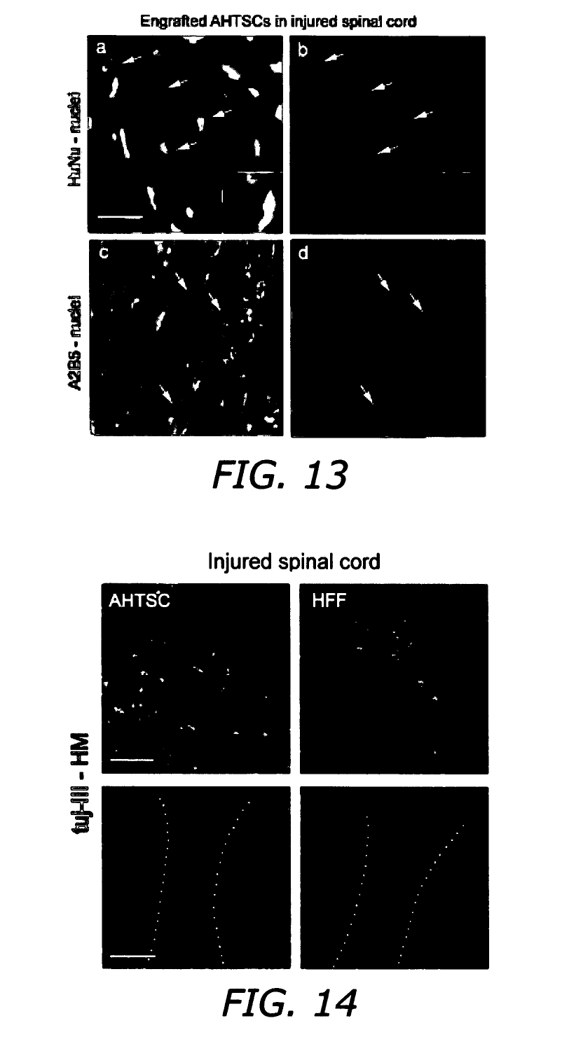

Cells from Example 5 at passage 2-3 were subjected to a brief neural differentiation for 4 days and transplanted into the spinal cord of NOD/SCID mice (n=4). AHTSCs were examined 3 weeks following transplantation (FIG. 11). Staining for human-specific nuclear proteins (HuNu) demonstrated that many AHTSCs survived and integrated into both the white and grey matter of the host tissue (FIGS. 11A and 11C, respectively). Confocal microscopy demonstrated that some AHTSCs were positive for neuronal marker Tuj-III (FIGS. 11A and 11B) but none was positive for the astroglial marker, GFAP (FIGS. 11C and D).

AHTSCs, human foreskin fibroblasts (HFF), and vehicle control (DMEM) (n=6 per group) were transplanted into NOD/SCID mice following spinal cord injury and the cords were evaluated 35 days post transplantation. Mice were anesthetized and transcardially perfused with 60 ml of 4% paraformaldehyde. Spinal cords were dissected out of the vertebral column and postfixed overnight in 4% paraformaldehyde. Cords were then placed into 25% sucrose/PBS until sunk. A block of cord from T4 to L3 was embedded longitudinally in OCT compound and frozen at −20° C. for sectioning. 10 μm thick longitudinal sections were serially placed onto slides as to each section on one slide was 200 μm apart from the following on that same slide.

Some sections were stained with Harris' hematoxylin and counterstained in 1% eosin. For morphometric analysis (n=6 per group), Hematoxylin and eosin (H&E) stained tissue sections were projected upon a computer screen at 40× magnification. Measurements of the diameter of the spinal cord at 100 μm intervals extending 1.0 mm rostral and 1.0 mm caudal to the injury epicenter were made using Olympus Microsuite™-Five basic edition analysis software (Olympus), using all tissue sections in which the central canal was visible. The tissue diameters at each 100 μm interval for each animal within a group were averaged.

For immunohistochemistry, sections were incubated overnight in either rabbit anti-MAP2 (Chemicon), rabbit anti-β-Tubulin III (Sigma), rabbit anti-GFAP (Chemicon), rabbit anti-NG2 (Chemicon), rabbit-anti-GalC (Sigma), mouse-anti-A2B5 (Chemicon), and mouse anti-human nuclei, or mouse anti-human mitochondria, (Chemicon) antibodies. Visualization was achieved by using AlexaFluor™ 488 goat anti-mouse, and/or AlexaFluor™ 568 goat anti-rabbit (Molecular Probes). All sections were counterstained with Hoescht 33342 or TO-PRO-3 (Molecular Probes).

Transplanted human cells were identified by staining of human nuclei (HuNu; FIG. 12). Many AHTSCs were found in or at the vicinity of the transplantation site (FIGS. 12A, 12C, 12E and 12G, low magnification). The AHTSC expressing oligodendroglial or neuronal markers was identified by double staining of NG2/HuNu, GalC/HuNu, Tuj-III/HuNu or MAP-2/HuNu (indicated by arrows in FIGS. 12B, 12D, 12F and 12H, in higher magnification).

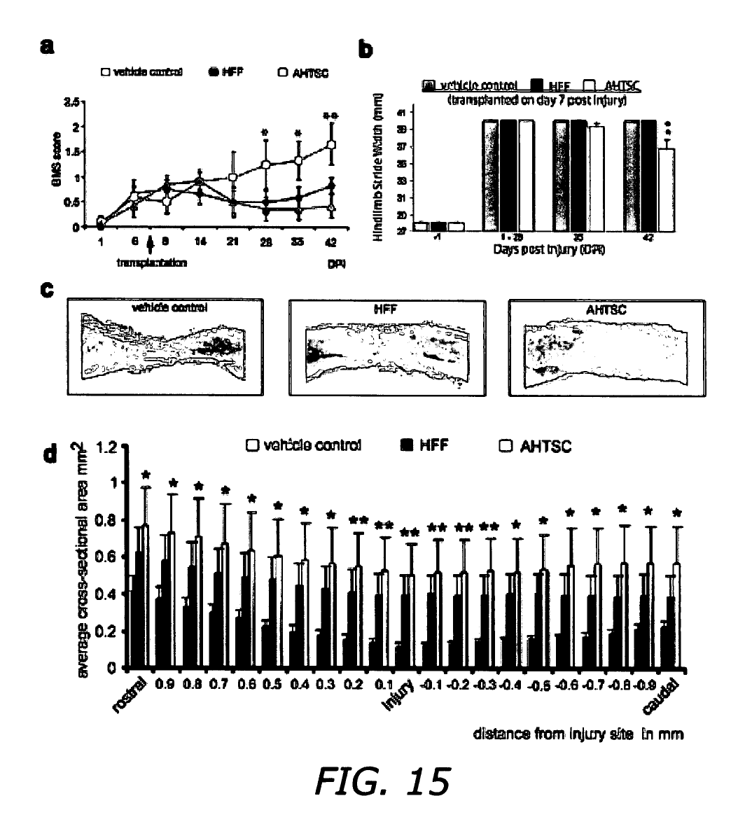

Using the Basso Mouse Scale, a significant reduction in functional deficits was observed for the AHTSCs transplanted cohort as compared to the HFF and vehicle control transplanted cohorts at days 28 (p<0.05), 35 (p<0.05), and 42 (p<0.01) following SCI (FIG. 15A). Moreover, kinematic analysis was also used to assess the functional recovery and found that hindlimb stride width was clearly improved (p<0.05) in the AHTSC cohort at 42 days post SCI, as compared to both vehicle control and HFF cohorts (FIG. 15B).

Morphometric analysis of H & E stained spinal cord sections indicated that transplantation with neural-enriched AHTSCs and HFF significantly (p<0.05) reduced tissue loss 1 mm either side of the injury epicenter as compared to vehicle control 35 days following SCI (FIGS. 15C and 15D). Abundant tissue loss around the injury site was evident in spinal cords from vehicle control mice as compared to cellular transplanted mice viewed under low magnification (FIG. 15C). There was no significant difference (p>0.1) in tissue loss for the neural-enriched AHTSCs transplanted cohort as compared to the HFF transplanted cohort. The average cross-sectional area 1 mm rostral and caudal to the injury epicenter was 0.586±0.19 mm² for the neural-enriched AHTSC transplanted cohort, 0.441±0.11 mm² for the HFF transplanted cohort, and 0.210±0.02 mm² for the vehicle control cohort. These data clearly demonstrate a significant reduction (p<0.05) in tissue loss after transplantation of the neural-enriched AHTSC or HFF cohort.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A method for therapeutic reprogramming comprising:
isolating a human adult stem cell;
contacting said human adult stem cell with a medium comprising stimulatory factors which induce development of said stem cell into a therapeutically reprogrammed cell;
recovering said therapeutically reprogrammed cell from said medium.

2. The method of claim 1 wherein said human adult stem cell is isolated from the testes.

3. The method of claim 2 wherein said human adult stem cell is a spermatogonial stem cell.

4. The method of claim 1 wherein said medium comprises PM-10™ medium.

5. The method of claim 1 wherein said therapeutically reprogrammed cell is matured into a more terminally differentiated cell.

6. The method of claim 5 wherein said more terminally differentiated cell is a cardiac myocyte.

7. The method of claim 5 wherein said more terminally differentiated cell is a neural cell.

8. The method of claim 1 further comprising the step of culturing said therapeutically reprogrammed cell to form a cell line.

9. The method of either of claims 1 or 8 further comprising the step of implanting said therapeutically reprogrammed cell, or a cell matured therefrom, into a host in need of a therapeutically reprogrammed cell.

10. A pluripotent therapeutic composition comprising a therapeutically reprogrammed human adult stem cell.

11. The pluripotent therapeutic composition of claim 10 wherein said human adult stem cell is isolated from the testes.

12. The pluripotent therapeutic composition of claim 10 wherein said therapeutically reprogrammed human adult stem cell is produced according to the therapeutic reprogramming method of claim 1.

13. A pluripotent therapeutic composition comprising a therapeutically reprogrammed human adult stem cell which has been induced into a more terminally differentiated cell prior to implantation into a host in need of a therapeutically reprogrammed cells.
